(19) United States
(12) Patent Application Publication
(10) Pub. No.: US 2024/0024162 A1
(43) Pub. Date: Jan. 25, 2024

(54) Ear Plug Earbud Tip Device

(71) Applicant: Chad Green, Cassville, MO (US)

(72) Inventor: Chad Green, Cassville, MO (US)

(21) Appl. No.: 18/355,818

(22) Filed: Jul. 20, 2023

Related U.S. Application Data
(60) Provisional application No. 63/391,221, filed on Jul. 21, 2022.

(51) Int. Cl. A61F 11/08

(52) U.S. Cl. CPC A61F 11/085

(57) ABSTRACT

An ear plug earbud tip device is provided. The device is comprised of an inner core positioned within an outer body, wherein the inner core has an opening that receives and mechanically snaps onto a tip of an earbud and allows sound to flow through the inner core and into the ear of a user. The inner core is surrounded by an outer body that surrounds an earbud body. The outer surface of the outer body may be comprised of a protrusion that secures the body within the ear of a user. During use, the shape of the device eliminates or reduces harmful background noise from entering the ear canal of a user.

### CROSS-REFERENCE TO RELATED APPLICATION

The present application claims priority to, and the benefit of, U.S. Provisional Application No. 63/391,221, which was filed on Jul. 21, 2022, and is incorporated herein by reference in its entirety.

### FIELD OF THE INVENTION

The present invention relates generally to the field of ear plugs. More specifically, the present invention relates to an ear plug earbud tip device that can be attached to the tip of an earbud and inserted into the ear of a user, wherein the device reduces background noise levels to a lower and safer threshold. This prevents the need for a user to increase the volume of an earbud device to potentially unsafe levels in an effort to overcome said elevated background noise pollution, just to hear the audio contents clearly. Accordingly, the present disclosure makes specific reference thereto. Nonetheless, it is to be appreciated that aspects of the present invention are also equally applicable to other like applications, devices, and methods of manufacture. It is to be noted that this invention and its variants utilized the method of passive “noise isolation”, not active “noise cancelation”.

### BACKGROUND

Many individuals enjoy using earbuds, headphones, etc. to listen to their favorite music, videos, podcasts, and other audio and media. An individual may listen to said audio while around excessive environmental background noise. An individual may also utilize said devices for communication purposes, such as phone calls and radio comms, also while around excessive environmental background noise. Examples of loud areas and activities include but are not limited to, performing lawn care activities, flying in aircraft, operating heavy equipment, communicating in construction zones, etc. When in loud environments, said individuals must then increase the volume of the listening device, sometimes drastically, just to overcome ambient background noise and hear their desired audio. This may result in poor audio clarity, distortion, a poor listening experience, unsafe volume levels for the user, potential hearing damage and loss, a lack of situational awareness and a greater potential for accidents and injury. The lower the volume levels one may communicate and enjoy audio input at, the safer they and their hearing may be.

Therefore, there exists a long-felt need in the art for an improved listening device. More specifically, there exists a long-felt need in the art for an ear plug earbud tip device. There also exists a long-felt need in the art for an earplug earbud tip device that can be applied to an earbud tip to reduce background noise while listening to the earbud. In addition, there exists a long-felt need in the art for an ear plug earbud tip device that can be applied to an earbud tip to reduce background noise while listening to the earbud and wherein the device can be universally applied to any earbud. Further, there exists a long-felt need in the art for an earplug earbud tip device that can be applied to an earbud tip to reduce background noise while listening to the earbud and wherein the device lowers background noise to hearing-safe levels.

The subject matter disclosed and claimed herein, in one embodiment thereof, comprises an ear plug earbud tip device. The device is comprised of an ear plug earbud tip device comprised of an inner core positioned within an outer body. The inner core has an opening that receives a tip of an earbud and allows sound to flow through the inner core and into a user's ear. The inner core is surrounded by an outer body that receives the earbud body. The outer surface of the outer body may be comprised of a protrusion that secures the body within the ear of a user. During use, the shape of the device eliminates or reduces harmful background noise from entering the ear canal of a user. As a result, a user need not increase the volume of an earbud to overcome said background noise.

In this manner, the ear plug earbud tip device of the present invention accomplishes all the forgoing objectives and provides an ear plug earbud tip device that can be applied to an earbud tip to reduce background noise while listening to the earbud. In addition, the device reduces background noise while listening to the earbud and wherein the device lowers background noise to hearing-safe levels. In this manner, the device improves the hearing safety of the user by allowing the user to listen to audio at a lower volume level. In addition, the device protects a user's ears from harmful levels of background noise.

### SUMMARY

The following presents a simplified summary to provide a basic understanding of some aspects of the disclosed innovation. This summary is not an extensive overview, and it is not intended to identify key/critical elements or to delineate the scope thereof. Its sole purpose is to present some general concepts in a simplified form as a prelude to the more detailed description that is presented later.

The subject matter disclosed and claimed herein, in one embodiment thereof, comprises an ear plug earbud tip device. The device is primarily comprised of an inner core that receives a tip of an earbud, wherein the inner core is positioned within an outer body. The device can be applied to the ear tip of an earbud to reduce background noises while allowing users to listen to the earbuds at a safe volume (i.e., without having to increase the volume of the earbud to an unsafe hearing level to overcome the background noise).

The inner core is preferably conical in shape (i.e., tapered) and is comprised of an opening that receives the tip of an earbud. The inner core is surrounded by an outer body comprised of a first opening that receives the inner core. The body is also comprised of a second opening positioned above the first opening that receives the earbud body when the device is attached to the earbud.

The outer surface of the body may be smooth in one embodiment. In another embodiment, the outer surface is comprised of at least one protrusion that may be round, capped, capped and tiered, jagged, or any shape that improves retention of the outer body within the ear canal of the user. The body is preferably made of a foam and or a silicone material such that it can be easily inserted into the ear canal of a user.

The present invention is also comprised of a method of using the device. First, a device is provided comprised of an inner core comprised of an opening and an outer body comprised of a first opening and a second opening, wherein the inner core is positioned within the outer body. Then, a tip of an earbud can be inserted into the inner core. Next, the body of the earbud can be inserted into the second opening of the outer body. Then, the device and earbud can be placed into the ear of the user and the earbud can be used to play audio into the ear of the user, wherein the device reduces background noise from the user's environment.

Another method of using the device is also provided. First, a device is provided comprised of an inner core comprised of an opening and an outer body comprised of a first opening and a second opening, wherein the inner core is positioned within the outer body. Next, the inner core can be placed inside the outer body via at least one opening and secured within the outer body via at least one fastener. Then, a tip of an earbud can be inserted into the inner core. Next, the body of the earbud can be inserted into the second opening of the outer body. Then, the device and earbud can be placed into the ear of the user and the earbud can be used to play audio into the ear of the user, wherein the device reduces background noise from the user's environment.

Accordingly, the ear plug earbud tip device of the present invention is particularly advantageous as it provides an ear plug earbud tip device that can be applied to an earbud tip to reduce background noise while listening to the earbud. In addition, the device reduces background noise while listening to the earbud and wherein the device lowers background noise to hearing-safe levels. In this manner, the device improves the hearing safety of the user by allowing the user to listen to audio at a lower volume level. In addition, the device protects a user's ears from harmful levels of background noise. As a result, the earplug earbud tip device overcomes the limitations of existing earbuds and earbud tips known in the art.

To the accomplishment of the foregoing and related ends, certain illustrative aspects of the disclosed innovation are described herein in connection with the following description and the annexed drawings. These aspects are indicative, however, of but a few of the various ways in which the principles disclosed herein can be employed and are intended to include all such aspects and their equivalents. Other advantages and novel features will become apparent from the following detailed description when considered in conjunction with the drawings.

### BRIEF DESCRIPTION OF THE DRAWINGS

The description refers to provided drawings in which similar reference characters refer to similar parts throughout the different views, and in which:

FIG. 1 illustrates an exploded and cross-sectional view of one potential embodiment of an ear plug earbud tip device of the present invention in accordance with the disclosed architecture;

FIG. 2 illustrates a cross-sectional view of one potential embodiment of an ear plug earbud tip device of the present invention in accordance with the disclosed architecture;

FIG. 3 illustrates a perspective view of one potential embodiment of an ear plug earbud tip device of the present invention in accordance with the disclosed architecture;

FIG. 4 illustrates a flowchart of a method of using one potential embodiment of an ear plug earbud tip device of the present invention in accordance with the disclosed architecture; and

FIG. 5 illustrates a flowchart of a method of using one potential embodiment of an ear plug earbud tip device of the present invention in accordance with the disclosed architecture.

### DETAILED DESCRIPTION

The innovation is now described with reference to the drawings, wherein like reference numerals are used to refer to like elements throughout. In the following description, for purposes of explanation, numerous specific details are set forth to provide a thorough understanding thereof. It may be evident, however, that the innovation can be practiced without these specific details. In other instances, well-known structures and devices are shown in block diagram form to facilitate a description thereof. Various embodiments are discussed hereinafter. It should be noted that the figures are described only to facilitate the description of the embodiments. They are not intended as an exhaustive description of the invention and do not limit the scope of the invention. Additionally, an illustrated embodiment need not have all the aspects or advantages shown. Thus, in other embodiments, any of the features described herein from different embodiments may be combined.

As noted above, there exists a long-felt need in the art for an improved listening device. More specifically, there exists a long-felt need in the art for an ear plug earbud tip device. There also exists a long-felt need in the art for an earplug earbud tip device that can be applied to an earbud tip to reduce background noise while listening to the earbud. In addition, there exists a long-felt need in the art for an ear plug earbud tip device that can be applied to an earbud tip to reduce background noise while listening to the earbud and wherein the device can be universally applied to any earbud. Further, there exists a long-felt need in the art for an earplug earbud tip device that can be applied to an earbud tip to reduce background noise while listening to the earbud and wherein the device lowers background noise to hearing-safe levels.

The present invention, in one exemplary embodiment, is comprised of an ear plug earbud tip device primarily comprised of an inner core that receives a tip of an earbud, wherein the inner core is positioned within an outer body. During use, the device can be applied to the ear tip of an earbud to reduce background noises while allowing users to listen to the earbuds at a safe volume (i.e., without having to increase the volume of the earbud to an unsafe hearing level to overcome the background noise).

The inner core is preferably conical in shape (i.e., tapered) and is comprised of an opening that receives the tip of an earbud. The inner core is surrounded by an outer body comprised of a first opening that receives the inner core. The body is also comprised of a second opening positioned above the first opening that receives the earbud body when the device is attached to the earbud. The outer surface of the body may be smooth in one embodiment or may be comprised of at least one protrusion that may be round, capped, capped and tiered, jagged, or any shape that improves retention of the outer body within the ear canal of the user. The body is preferably made of a foam material such that it can be easily inserted into the ear canal of a user.

The present invention is also comprised of a method of using the device. First, a device is provided comprised of an inner core comprised of an opening and an outer body comprised of a first opening and a second opening, wherein the inner core is positioned within the outer body. Then, a tip of an earbud can be inserted into the inner core. Next, the body of the earbud can be inserted into the second opening of the outer body. Then, the device and earbud can be placed into the ear of the user and the earbud can be used to play audio into the ear of the user, wherein the device reduces background noise from the user's environment.

Another method of using the device is also provided. First, a device is provided comprised of an inner core comprised of an opening and an outer body comprised of a first opening and a second opening, wherein the inner core is positioned within the outer body. Next, the inner core can be placed inside the outer body via at least one opening and secured within the outer body via at least one fastener. Then, a tip of an earbud can be inserted into the inner core. Next, the body of the earbud can be inserted into the second opening of the outer body. Then, the device and earbud can be placed into the ear of the user and the earbud can be used to play audio into the ear of the user, wherein the device reduces background noise from the user's environment.

Accordingly, the ear plug earbud tip device of the present invention is particularly advantageous as it provides an ear plug earbud tip device that can be applied to an earbud tip to reduce background noise while listening to the earbud. In addition, the device reduces background noise while listening to the earbud and wherein the device lowers background noise to hearing-safe levels. In this manner, the device improves the hearing safety of the user by allowing the user to listen to audio at a lower volume level. In addition, the device protects a user's ears from harmful levels of background noise. As a result, the earplug earbud tip device overcomes the limitations of existing earbuds and earbud tips known in the art.

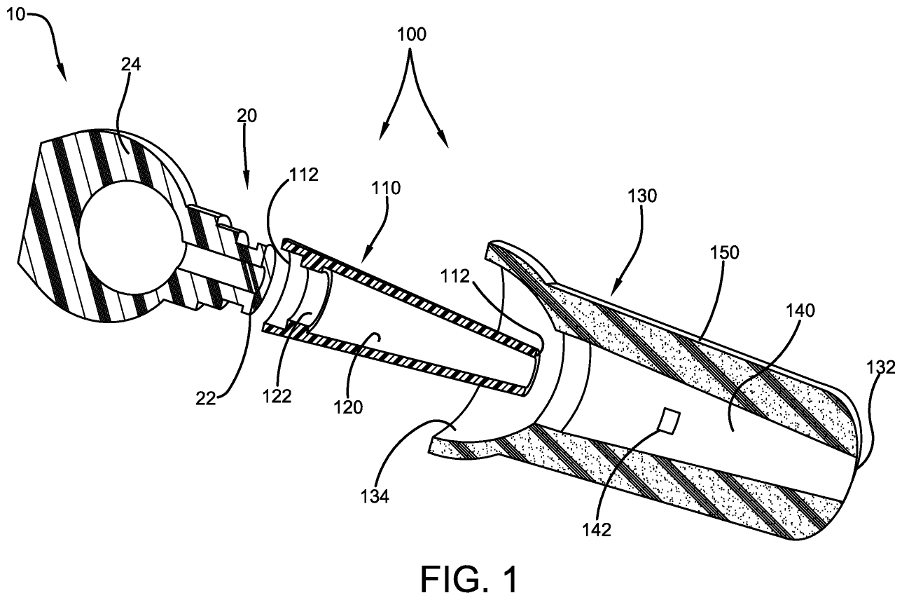

Referring initially to the drawings, FIG. 1 illustrates an exploded and cross-sectional view of one potential embodiment of an ear plug earbud tip device 100 of the present invention in accordance with the disclosed architecture. The device 100 is primarily comprised of an inner core 110 that receives a tip 20 of an earbud 10, wherein the inner core 110 is positioned within an outer body 130. The device 100 can be applied to the ear tip 20 of an earbud 10 to reduce background noises (preferably by at least 10 to 30 decibels) while allowing users to listen to the earbuds 10 at a safe volume (i.e., without having to increase the volume of the earbud 10 to an unsafe hearing level to overcome the background noise).

The inner core 110 is preferably made from a silicone material. In addition, the outer body 130 is preferably comprised of a foam material. However, the inner core 110 and outer body 130 may be made from any earbud or earplug material of the like.

The inner core 110 is preferably conical in shape (i.e., tapered). The inner core 110 is comprised of an opening 112. The opening 112 receives the tip 20 of an earbud 10. More specifically, the interior surface 120 of the inner core 110 is comprised of at least one recessed or raised channel/lip 122 that runs radially a portion of the interior surface 120. The channel 122 receives a T-shape member 22 of an earbud tip 20. It should be appreciated that many existing earbuds 10 known in the art have a plurality of tip 20 styles. Accordingly, the inner core 110 may be designed with an interior surface 120 and/or channel 122 (or other structure of the like such as but not limited to a protruding lock ring, protrusion, recession, etc.) that receives and secures any style of earbud tip 20 known in the art. The opening 112 also allows sound from the earbud 10 to travel into the ear of the user.

The inner core 110 is surrounded by an outer body 130. More specifically, the body 130 is comprised of a first opening 132 that receives the inner core 110. The interior surface 140 of the body 130 may be comprised of a fastener 142 to secure the to secure the tip of inner core 110 flush with the end of outer body 130 at opening 132. This allows a user to interchange a plurality of inner core 110 and outdoor body 130 shapes/sizes for optimal fit and noise reduction. In another embodiment, the core 110 is integrally formed within the body 130, wherein each 110,130 may be made of the same or different material.

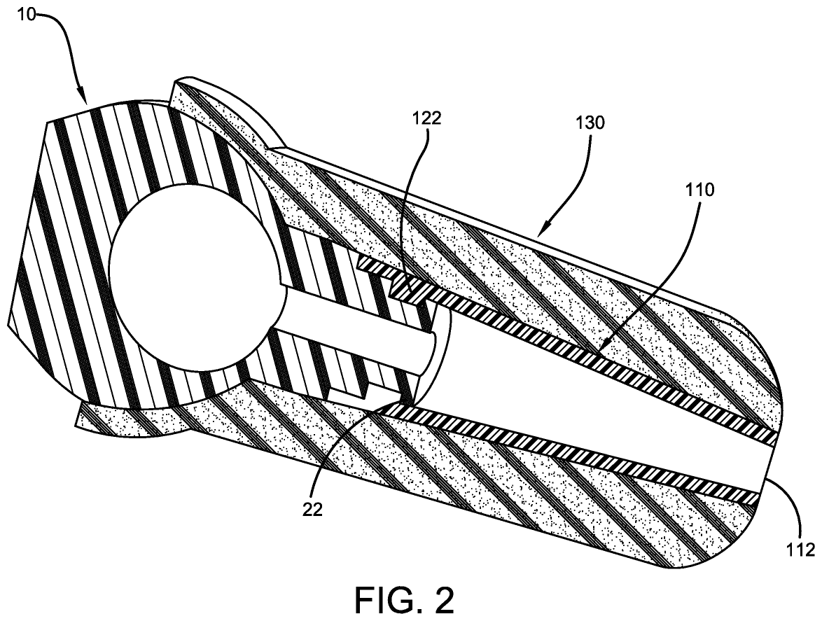

The body 130 is also comprised of a second opening 134 positioned above the first opening 132. The second opening 134 receives the earbud 10 body 24 when the device 100 is attached to the earbud 10, as seen in FIG. 2. This provides comfort for the user by surrounding the earbud 10 body 24. However, depending on the style of earbud 10 the device 100 is applied to, the device 100 may not cover the body 24 of the earbud 10 such that the opening 134 may not extend around the earbud 10 body 24 and instead only allows sound to travel through the device 100 (i.e., no portion of the device 10 covers the body 24 of the earbud 10).

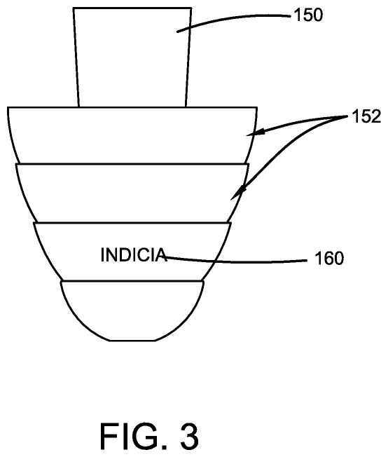

The outer surface 150 of the body 130 may be smooth in one embodiment. In another embodiment, the outer surface 150 is comprised of at least one protrusion 152, as seen in FIG. 3. The protrusions may be round, capped, capped and tiered, jagged, or any shape that improves retention of the outer body 130 within the ear canal of the user. The body 130 is preferably made of a foam material such that it can be easily inserted into the ear canal of a user. However, the body 130 may alternatively be made from a silicone or other material of the like. The body 130 may be comprised of at least one indicia 160 that may be in the form of a pattern, design, picture, letter, number, symbol, logo, etc. for aesthetic purposes.

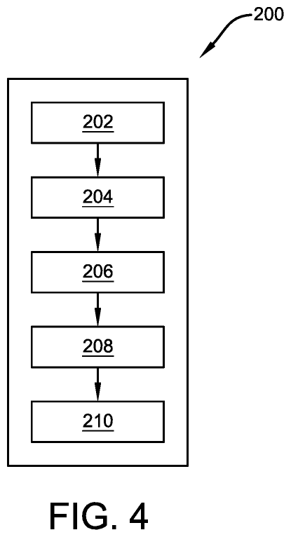

The present invention is also comprised of a method of using 200 the device 100, as seen in FIG. 4. First, a device 100 is provided comprised of an inner core 110 comprised of an opening 112 and an outer body 130 comprised of a first opening 132 and a second opening 134, wherein the inner core 110 is positioned within the outer body 130 [Step 202]. Then, a tip 20 of an earbud 10 can be inserted into the inner core 110 [Step 204]. More specifically, a T-shaped member 22 of the tip 20 may be inserted into at least one channel 122 of the inner core 110. Then, the body 24 of the earbud 10 can be inserted into the second opening 134 of the outer body 130 [Step 206]. Next, the device 100 (and attached earbud 10) can be placed into the ear of the user [Step 208]. Then, the earbud 10 can be used to play audio into the ear of the user, wherein the device 100 reduces background noise from the user's environment [Step 210]. In an alternative embodiment, the device 100 can be placed in the ear of the user (Step 208) to allow the inner core 10 to expand wherein steps 202, 204, and 206 may then be performed.

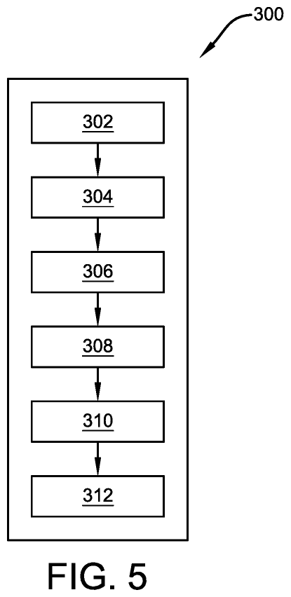

The present invention may also be comprised of a second method of using 300 the device 100, as seen in FIG. 5. First, a device 100 is provided comprised of an inner core 110 comprised of an opening 112 and an outer body 130 comprised of a first opening 132 and a second opening 134, wherein the inner core 110 is positioned within the outer body 130 [Step 302]. Then, the inner core 110 can be placed inside the outer body 130 via at least one opening 132 and secured within the outer body 130 via at least one fastener 142 [Step 304]. Then, a tip 20 of an earbud 10 can be inserted into the inner core 110 [Step 306]. More specifically, a T-shaped member 22 of the tip 20 may be inserted into at least one channel 122 of the inner core 110. Then, the body 24 of the earbud 10 can be inserted into the second opening 134 of the outer body 130 [Step 308]. Next, the device 100 and earbud 10 can be placed into the ear of the user [Step 310]. Then, the earbud 10 can be used to play audio into the ear of the user, wherein the device 100 reduces background noise from the user's environment [Step 312]. In an alternative embodiment, the device 100 can be placed in the ear of the user (Step 308) to allow the inner core 10 to expand wherein steps 304, 306, and 308 may then be performed.

Certain terms are used throughout the following description and claims to refer to particular features or components. As one skilled in the art will appreciate, different persons may refer to the same feature or component by different names. This document does not intend to distinguish between components or features that differ in name but not structure or function. As used herein “ear plug earbud tip device” and “device” are interchangeable and refer to the earplug earbud tip device 100 of the present invention.

Notwithstanding the forgoing, the ear plug earbud tip device 100 of the present invention and its various components can be of any suitable size and configuration as is known in the art without affecting the overall concept of the invention, provided that they accomplish the above-stated objectives. One of ordinary skill in the art will appreciate that the size, configuration, and material of the ear plug earbud tip device 100 as shown in the FIGS. are for illustrative purposes only, and that many other sizes and shapes of the earplug earbud tip device 100 are well within the scope of the present disclosure. Although the dimensions of the earplug earbud tip device 100 are important design parameters for user convenience, the ear plug earbud tip device 100 may be of any size, shape, and/or configuration that ensures optimal performance during use and/or that suits the user's needs and/or preferences.

Various modifications and additions can be made to the exemplary embodiments discussed without departing from the scope of the present invention. While the embodiments described above refer to particular features, the scope of this invention also includes embodiments having different combinations of features and embodiments that do not include all the described features. Accordingly, the scope of the present invention is intended to embrace all such alternatives, modifications, and variations as fall within the scope of the claims, together with all equivalents thereof.

What has been described above includes examples of the claimed subject matter. It is, of course, not possible to describe every conceivable combination of components or methodologies for purposes of describing the claimed subject matter, but one of ordinary skill in the art may recognize that many further combinations and permutations of the claimed subject matter are possible. Accordingly, the claimed subject matter is intended to embrace all such alterations, modifications, and variations that fall within the spirit and scope of the appended claims. Furthermore, to the extent that the term “includes” is used in either the detailed description or the claims, such term is intended to be inclusive in a manner similar to the term “comprising” as “comprising” is interpreted when employed as a transitional word in a claim.

What is claimed is:

1. An ear plug earbud tip device comprising:
an outer body comprised of a first opening and a second opening; and
an inner core comprised of a third opening, wherein the inner core positioned within the outer body.

2. An ear plug earbud tip device of claim 1, wherein the inner core is comprised of a silicone material.

3. An ear plug earbud tip device of claim 1, wherein the outer body is comprised of a foam material.

4. An ear plug earbud tip device of claim 1, wherein the inner core is comprised of a tapered shape.

5. An ear plug earbud tip device of claim 1, wherein the inner core is fixedly attached to the outer body.

6. An ear plug earbud tip device of claim 1, wherein an outer surface of the outer body is comprised of a protrusion.

7. An ear plug earbud tip device of claim 6, wherein the protrusion is comprised of a round protrusion.

8. An ear plug earbud tip device of claim 6, wherein the protrusion is comprised of a capped protrusion.

9. An ear plug earbud tip device of claim 6, wherein the protrusion is comprised of a capped and tiered protrusion.

10. An ear plug earbud tip device of claim 6, wherein the protrusion is comprised of a jagged protrusion.

11. An ear plug earbud tip device of claim 6, wherein the first opening is larger than the second opening.

12. An ear plug earbud tip device comprising:
an outer body comprised of a first opening, a second opening, and a protrusion; and
an inner core comprised of a third opening and a channel, the inner core positioned within the outer body.

13. An ear plug earbud tip device of claim 12, wherein the channel is positioned on an interior surface of the inner core.

14. An ear plug earbud tip device of claim 13, wherein the channel is positioned radially around the interior surface.

15. An ear plug earbud tip device of claim 12, wherein an interior surface of the outer body is comprised of a fastener.

16. An ear plug earbud tip device of claim 12, wherein the first opening is larger than the second opening.

17. An ear plug earbud tip device of claim 12, wherein the protrusion is positioned on an outer surface of the outer body.

18. An ear plug earbud tip device of claim 17, wherein the protrusion is comprised of a round protrusion, a capped protrusion, a capped and tiered protrusion, or a jagged protrusion.

19. A method of using an ear plug earbud tip device, the method comprising the following steps:
providing an ear plug earbud tip device comprised of an outer body comprised of a first opening and a second opening, wherein an inner body comprised of a third opening is positioned within the outer body;
placing a tip of an earbud into the inner core;
inserting a body of the earbud into the second opening;
placing the earbud and the attached ear plug earbud tip device into an ear; and
listening to an audio from the earbud.

20. A method of using an ear plug earbud tip device of claim 19, wherein the tip of the earbud is received by a channel positioned on an interior surface of the inner core.
